SEQUENCE LISTING (119 sequences)

SEQ ID NO: 1 - DNA, 34 bp, Artificial Sequence
  primer FcM202Lss
cttctcatgctccgtgctgcatgaggctctgcac

SEQ ID NO: 2 - DNA, 34 bp, Artificial Sequence
  primer FcM202Las
gtgcagagcctcatgcagcacggagcatgagaag

SEQ ID NO: 3 - DNA, 33 bp, Artificial Sequence
  primer FcN208Sss
catgaggctctgcacagccactacacacagaag

SEQ ID NO: 4 - DNA, 33 bp, Artificial Sequence
  primer FcN208Sas
cttctgtgtgtagtggctgtgcagagcctcatg

SEQ ID NO: 5 - DNA, 34 bp, Artificial Sequence
  primer FcI27Fss
caaggacaccctcatgttctcccggacccctgag

SEQ ID NO: 6 - DNA, 34 bp, Artificial Sequence
  primer FcI27Fas
ctcaggggtccgggagaacatgagggtgtccttg

SEQ ID NO: 7 - DNA, 34 bp, Artificial Sequence
  primer FcH207Tss
gatgcatgaggctctgaccaaccactacacacag

SEQ ID NO: 8 - DNA, 34 bp, Artificial Sequence
  primer FcH207Tas
ctgtgtgtagtggttggtcagagcctcatgcatc

SEQ ID NO: 9 - DNA, 34 bp, Artificial Sequence
  primer FcH207Kss
gatgcatgaggctctgaagaaccactacacacag

SEQ ID NO: 10 - DNA, 34 bp, Artificial Sequence
  primer FcH207Kas
ctgtgtgtagtggttcttcagagcctcatgcatc

SEQ ID NO: 11 - DNA, 34 bp, Artificial Sequence
  primer FcL83Fss
cagcgtcctcaccgtcttccaccaggactggctg

SEQ ID NO: 12 - DNA, 34 bp, Artificial Sequence
  primer FcL83Fas
cagccagtcctggtggaagacggtgaggacgctg

SEQ ID NO: 13 - DNA, 34 bp, Artificial Sequence
  primer FcI27Lss
caaggacaccctcatgctctcccggacccctgag

SEQ ID NO: 14 - DNA, 34 bp, Artificial Sequence
  primer FcI27Las
ctcaggggtccgggagagcatgagggtgtccttg

SEQ ID NO: 15 - DNA, 34 bp, Artificial Sequence
  primer FcH207K/N208Sss
gatgcatgaggctctgaagagccactacacacag

SEQ ID NO: 16 - DNA, 34 bp, Artificial Sequence
  primer FcH207K/N208Sas
ctgtgtgtagtggctcttcagagcctcatgcatc

SEQ ID NO: 17 - DNA, 33 bp, Artificial Sequence
  primer FcN208Tss
catgaggctctgcacacccactacacacagaag

SEQ ID NO: 18 - DNA, 33 bp, Artificial Sequence
  primer FcN208Tas
cttctgtgtgtagtgggtgtgcagagcctcatg

SEQ ID NO: 19 - DNA, 34 bp, Artificial Sequence
  primer FcH207T/N208Sss
gatgcatgaggctctgaccagccactacacacag

SEQ ID NO: 20 - DNA, 34 bp, Artificial Sequence
  primer FcH207T/N208Sas
ctgtgtgtagtggctggtcagagcctcatgcatc

SEQ ID NO: 21 - DNA, 34 bp, Artificial Sequence
  primer FcE204Rss
atgctccgtgatgcatcgggctctgcacaaccac

SEQ ID NO: 22 - DNA, 34 bp, Artificial Sequence
  primer FcE204Ras
gtggttgtgcagagcccgatgcatcacggagcat

SEQ ID NO: 23 - DNA, 34 bp, Artificial Sequence
  primer FcE204Kss
atgctccgtgatgcataaggctctgcacaaccac

SEQ ID NO: 24 - DNA, 34 bp, Artificial Sequence
  primer FcE204Kas
gtggttgtgcagagccttatgcatcacggagcat

SEQ ID NO: 25 - DNA, 42 bp, Artificial Sequence
  primer FcE204R/H207Kss
tgctccgtgatgcatcgggctctgaagaaccactacacacag

SEQ ID NO: 26 - DNA, 42 bp, Artificial Sequence
  primer FcE204R/H207Kas
ctgtgtgtagtggttcttcagagcccgatgcatcacggagca

SEQ ID NO: 27 - DNA, 42 bp, Artificial Sequence
  primer FcE204K/H207Kss
tgctccgtgatgcataaggctctgaagaaccactacacacag

SEQ ID NO: 28 - DNA, 42 bp, Artificial Sequence
  primer FcE204K/H207Kas
ctgtgtgtagtggttcttcagagccttatgcatcacggagca

SEQ ID NO: 29 - DNA, 34 bp, Artificial Sequence
  primer FcH203Rss
ctcatgctccgtgatgcgtgaggctctgcacaac

SEQ ID NO: 30 - DNA, 34 bp, Artificial Sequence
  primer FcH203Ras
gttgtgcagagcctcacgcatcacggagcatgag

SEQ ID NO: 31 - DNA, 34 bp, Artificial Sequence
  primer FcH203Kss
ctcatgctccgtgatgaaggaggctctgcacaac

SEQ ID NO: 32 - DNA, 34 bp, Artificial Sequence
  primer FcH203Kas
gttgtgcagagcctccttcatcacggagcatgag

SEQ ID NO: 33 - DNA, 44 bp, Artificial Sequence
  primer FcH203R/H207Kss
catgctccgtgatgcgtgaggctctgaagaaccactacacacag

SEQ ID NO: 34 - DNA, 44 bp, Artificial Sequence
  primer FcH203R/H207Kas
ctgtgtgtagtggttcttcagagcctcacgcatcacggagcatg

SEQ ID NO: 35 - DNA, 44 bp, Artificial Sequence
  primer FcH203K/H207Kss
catgctccgtgatgaaggaggctctgaagaaccactacacacag

SEQ ID NO: 36 - DNA, 44 bp, Artificial Sequence
  primer FcH203K/H207Kas
ctgtgtgtagtggttcttcagagcctccttcatcacggagcatg

SEQ ID NO: 37 - DNA, 49 bp, Artificial Sequence
  primer FcM202L/H207Kss
cttctcatgctccgtgctgcatgaggctctgaagaaccactacacacag

SEQ ID NO: 38 - DNA, 49 bp, Artificial Sequence
  primer FcM202L/H207Kas
ctgtgtgtagtggttcttcagagcctcatgcagcacggagcatgagaag

SEQ ID NO: 39 - DNA, 49 bp, Artificial Sequence
  primer FcH203K/N208Tss
ctcatgctccgtgatgaaggaggctctgcacacccactacacacagaag

SEQ ID NO: 40 - DNA, 49 bp, Artificial Sequence
  primer FcH203K/N208Tas
cttctgtgtgtagtgggtgtgcagagcctccttcatcacggagcatgag

SEQ ID NO: 41 - DNA, 33 bp, Artificial Sequence
  primer FcT81Ess
gtggtcagcgtcctcgaagtcctgcaccaggac

SEQ ID NO: 42 - DNA, 33 bp, Artificial Sequence
  primer FcT81Eas
gtcctggtgcaggacttcgaggacgctgaccac

SEQ ID NO: 43 - DNA, 33 bp, Artificial Sequence
  primer FcE154Ass
agcgacatcgccgtggcgtgggagagcaatggg

SEQ ID NO: 44 - DNA, 33 bp, Artificial Sequence
  primer FcE154Aas
cccattgctctcccacgccacggcgatgtcgct

SEQ ID NO: 45 - DNA, 33 bp, Artificial Sequence
  primer FcE154Sss
agcgacatcgccgtgtcgtgggagagcaatggg

SEQ ID NO: 46 - DNA, 33 bp, Artificial Sequence
  primer FcE154Sas
cccattgctctcccacgacacggcgatgtcgct

SEQ ID NO: 47 - DNA, 33 bp, Artificial Sequence
  primer FcV82Pss
gtcagcgtcctcacccccctgcaccaggactgg

SEQ ID NO: 48 - DNA, 33 bp, Artificial Sequence
  primer FcV82Pas
ccagtcctggtgcaggggggtgaggacgctgac

SEQ ID NO: 49 - DNA, 33 bp, Artificial Sequence
  primer FcE204Sss
tgctccgtgatgcattcggctctgcacaaccac

SEQ ID NO: 50 - DNA, 33 bp, Artificial Sequence
  primer FcE204Sas
gtggttgtgcagagccgaatgcatcacggagca

SEQ ID NO: 51 - DNA, 33 bp, Artificial Sequence
  primer FcT81Sss
gtggtcagcgtcctctccgtcctgcaccaggac

SEQ ID NO: 52 - DNA, 33 bp, Artificial Sequence
  primer FcT81Sas
gtcctggtgcaggacggagaggacgctgaccac

SEQ ID NO: 53 - DNA, 34 bp, Artificial Sequence
  primer FcH203A/E204Sss
tcatgctccgtgatggcttcggctctgcacaacc

SEQ ID NO: 54 - DNA, 34 bp, Artificial Sequence
  primer FcH203A/E204Sas
ggttgtgcagagccgaagccatcacggagcatga

SEQ ID NO: 55 - DNA, 34 bp, Artificial Sequence
  primer FcH207A/N208Sss
gatgcatgaggctctggccagccactacacacag

SEQ ID NO: 56 - DNA, 34 bp, Artificial Sequence
  primer FcH207A/N208Sas
ctgtgtgtagtggctggccagagcctcatgcatc

SEQ ID NO: 57 - DNA, 32 bp, Artificial Sequence
  primer FcE204A/N208Sss
gctccgtgatgcatgcggctctgcacagccac

SEQ ID NO: 58 - DNA, 32 bp, Artificial Sequence
  primer FcE204A/N208Sas
gtggctgtgcagagccgcatgcatcacggagc

SEQ ID NO: 59 - DNA, 34 bp, Artificial Sequence
  primer FcH203A/N208Sss
ctcatgctccgtgatggctgaggctctgcacagc

SEQ ID NO: 60 - DNA, 34 bp, Artificial Sequence
  primer FcH203A/N208Sas
gctgtgcagagcctcagccatcacggagcatgag

SEQ ID NO: 61 - DNA, 34 bp, Artificial Sequence
  primer FcM202A/N208Sss
cttctcatgctccgtggcgcatgaggctctgcac

SEQ ID NO: 62 - DNA, 34 bp, Artificial Sequence
  primer FcM202A/N208Sas
gtgcagagcctcatgcgccacggagcatgagaag

SEQ ID NO: 63 - DNA, 34 bp, Artificial Sequence
  primer FcY93Lss
gctgaatggcaaggagctcaagtgcaaggtctcc

SEQ ID NO: 64 - DNA, 34 bp, Artificial Sequence
  primer FcY93Las
ggagaccttgcacttgagctccttgccattcagc

SEQ ID NO: 65 - DNA, 33 bp, Artificial Sequence
  primer FcV82Lss
ggtcagcgtcctcaccctcctgcaccaggactg

SEQ ID NO: 66 - DNA, 33 bp, Artificial Sequence
  primer FcV82Las
cagtcctggtgcaggagggtgaggacgctgacc

SEQ ID NO: 67 - DNA, 34 bp, Artificial Sequence
  primer FcT81Ass
gtgtggtcagcgtcctcgccgtcctgcaccagga

SEQ ID NO: 68 - DNA, 34 bp, Artificial Sequence
  primer FcT81Aas
tcctggtgcaggacggcgaggacgctgaccacac

SEQ ID NO: 69 - DNA, 34 bp, Artificial Sequence
  primer FcH207L/N208Sss
gatgcatgaggctctgctcagccactacacacag

SEQ ID NO: 70 - DNA, 34 bp, Artificial Sequence
  primer FcH207L/N208Sas
ctgtgtgtagtggctgagcagagcctcatgcatc

SEQ ID NO: 71 - DNA, 34 bp, Artificial Sequence
  primer FcY93A/N208Sss
gctgaatggcaaggaggccaagtgcaaggtctcc

SEQ ID NO: 72 - DNA, 34 bp, Artificial Sequence
  primer FcY93A/N208Sas
ggagaccttgcacttggcctccttgccattcagc

SEQ ID NO: 73 - protein, 221 aa, Artificial Sequence
  Synthetic HMC001 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 221 aa, Artificial Sequence
  Synthetic HMC002 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 75 - protein, 221 aa, Artificial Sequence
  Synthetic HMC003 Fc fragment
PSCPAPEFLGGPSVFLFPPKPKDTLMFSRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 221 aa, Artificial Sequence
  Synthetic HMC004 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALTNHYTQKSLSLSLGK

SEQ ID NO: 77 - protein, 221 aa, Artificial Sequence; identical to SEQ ID NO: 76
  Synthetic HMC005 Fc fragment

SEQ ID NO: 78 - protein, 221 aa, Artificial Sequence
  Synthetic HMC006 Fc fragment
PSCPAPEFLGGPSVFLFPPKPKDTLMISRTPEVTCVVVDVSQEDPEVQFNWYVDGVEVHNAKTKPREEQFNSTYRVVSVLTVFHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 79 - protein, 221 aa, Artificial Sequence
  Synthetic HMC007 Fc fragment
PSCPAPEFLGGPSVFLFPPKPKDTLMLSRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 221 aa, Artificial Sequence
  Synthetic HMC008 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALKSHYTQKSLSLSLGK

SEQ ID NO: 81 - protein, 221 aa, Artificial Sequence
  Synthetic HMC009 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 82 - protein, 221 aa, Artificial Sequence
  Synthetic HMC010 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALTSHYTQKSLSLSLGK

SEQ ID NO: 83 - protein, 221 aa, Artificial Sequence
  Synthetic HMC011 Fc fragment
PSCPAPEFLGGPSVFLFPPKPKDTLMISRTPEVTCVVVDVSQEDPEVQFNWYVDGVEVHNAKTKPREEQFNSTYRVVSVLTVFHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 221 aa, Artificial Sequence
  Synthetic HMC012 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHRALHNHYTQKSLSLSLGK

SEQ ID NO: 85 - protein, 221 aa, Artificial Sequence
  Synthetic HMC013 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHKALHNHYTQKSLSLSLGK

SEQ ID NO: 86 - protein, 221 aa, Artificial Sequence
  Synthetic HMC014 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHRALKNHYTQKSLSLSLGK

SEQ ID NO: 87 - protein, 221 aa, Artificial Sequence; identical to SEQ ID NO: 86
  Synthetic HMC015 Fc fragment

SEQ ID NO: 88 - protein, 221 aa, Artificial Sequence
  Synthetic HMC016 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMREALHNHYTQKSLSLSLGK

SEQ ID NO: 89 - protein, 221 aa, Artificial Sequence
  Synthetic HMC017 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMKEALHNHYTQKSLSLSLGK

SEQ ID NO: 90 - protein, 221 aa, Artificial Sequence
  Synthetic HMC018 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMREALKNHYTQKSLSLSLGK

SEQ ID NO: 91 - protein, 221 aa, Artificial Sequence
  Synthetic HMC019 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMKEALKNHYTQKSLSLSLGK

SEQ ID NO: 92 - protein, 221 aa, Artificial Sequence
  Synthetic HMC020 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALKNHYTQKSLSLSLGK

SEQ ID NO: 93 - protein, 221 aa, Artificial Sequence
  Synthetic HMC021 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMKEALHTHYTQKSLSLSLGK

SEQ ID NO: 94 - protein, 221 aa, Artificial Sequence
  Synthetic HMC022 Fc fragment
PSCPAPEFLGGPSVFLFPPKPKDTLMISRTPEVTCVVVDVSQEDPEVQFNWYVDGVEVHNAKTKPREEQFNSTYRVVSVLEVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 221 aa, Artificial Sequence
  Synthetic HMC023 Fc fragment
PSCPAPEFLGGPSVFLFPPKPKDTLMISRTPEVTCVVVDVSQEDPEVQFNWYVDGVEVHNAKTKPREEQFNSTYRVVSVLEVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 96 - protein, 221 aa, Artificial Sequence
  Synthetic HMC024 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVAWESNGQPENNYKTTPPVLDSDGSFFLYSRLTVDKSRWQEGNVFSCSVMHEALHNHYTQKSLSLSLGK

SEQ ID NO: 97 - protein, 221 aa, Artificial Sequence
  Synthetic HMC025 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVSWESNGQPENNYKTTPPVLDSDGSFFLYSRLTVDKSRWQEGNVFSCSVMHEALHNHYTQKSLSLSLGK

SEQ ID NO: 98 - protein, 221 aa, Artificial Sequence
  Synthetic HMC026 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVAWESNGQPENNYKTTPPVLDSDGSFFLYSRLTVDKSRWQEGNVFSCSVMHEALHSHYTQKSLSLSLGK

SEQ ID NO: 99 - protein, 221 aa, Artificial Sequence
  Synthetic HMC027 Fc fragment
PSCPAPEFLGGPSVFLFPPKPKDTLMISRTPEVTCVVVDVSQEDPEVQFNWYVDGVEVHNAKTKPREEQFNSTYRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHSALHSHYTQKSLSLSLGK

SEQ ID NO: 100 - protein, 221 aa, Artificial Sequence
  Synthetic HMC028 Fc fragment
PSCPAPEFLGGPSVFLFPPKPKDTLMISRTPEVTCVVVDVSQEDPEVQFNWYVDGVEVHNAKTKPREEQFNSTYRVVSVLTPLHQDWLNGKEYKCKVSNKGLPSSIEKTISKAKGQPREPQVYTLPPSQEEMTKNQVSLTCLVKGFYPSDIAVAWESNGQPENNYKTTPPVLDSDGSFFLYSRLTVDKSRWQEGNVFSCSVMHEALHNHYTQKSLSLSLGK

SEQ ID NO: 101 - protein, 221 aa, Artificial Sequence
  Synthetic HMC029 Fc fragment
PSCPAPEFLGGPSVFLFPPKPKDTLMISRTPEVTCVVVDVSQEDPEVQFNWYVDGVEVHNAKTKPREEQFNSTYRVVSVLSVLHQDWLNGKEYKCKVSNKGLPSSIEKTISKAKGQPREPQVYTLPPSQEEMTKNQVSLTCLVKGFYPSDIAVSWESNGQPENNYKTTPPVLDSDGSFFLYSRLTVDKSRWQEGNVFSCSVMHEALHNHYTQKSLSLSLGK

SEQ ID NO: 102 - protein, 221 aa, Artificial Sequence
  Synthetic HMC030 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMASALHNHYTQKSLSLSLGK

SEQ ID NO: 103 - protein, 221 aa, Artificial Sequence; identical to SEQ ID NO: 98
  Synthetic HMC031 Fc fragment

SEQ ID NO: 104 - protein, 221 aa, Artificial Sequence
  Synthetic HMC032 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALASHYTQKSLSLSLGK

SEQ ID NO: 105 - protein, 221 aa, Artificial Sequence
  HMC033 Fc fragment derivative
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHAALHSHYTQKSLSLSLGK

SEQ ID NO: 106 - protein, 221 aa, Artificial Sequence
  Synthetic HMC034 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMAEALHSHYTQKSLSLSLGK

SEQ ID NO: 107 - protein, 221 aa, Artificial Sequence
  Synthetic HMC035 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVAHEALHSHYTQKSLSLSLGK

SEQ ID NO: 108 - protein, 221 aa, Artificial Sequence
  Synthetic HMC036 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKELKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 109 - protein, 221 aa, Artificial Sequence
  Synthetic HMC037 Fc fragment
PSCPAPEFLGGPSVFLFPPKPKDTLMISRTPEVTCVVVDVSQEDPEVQFNWYVDGVEVHNAKTKPREEQFNSTYRVVSVLTLLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 110 - protein, 221 aa, Artificial Sequence
  Synthetic HMC038 Fc fragment
PSCPAPEFLGGPSVFLFPPKPKDTLMISRTPEVTCVVVDVSQEDPEVQFNWYVDGVEVHNAKTKPREEQFNSTYRVVSVLAVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 111 - protein, 221 aa, Artificial Sequence
  Synthetic HMC039 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALLSHYTQKSLSLSLGK

SEQ ID NO: 112 - protein, 221 aa, Artificial Sequence
  Synthetic HMC040 Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEAKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 113 - DNA, 663 bp, Artificial Sequence
  Synthetic HMC002 Fc fragment
ccatcatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgctgcatgaggctctgcacagccactacacacagaagagcctctccctgtctctgggtaaa

SEQ ID NO: 114 - DNA, 663 bp, Artificial Sequence
  Synthetic HMC008 Fc fragment
ccatcatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgatgcatgaggctctgaagagccactacacacagaagagcctctccctgtctctgggtaaa

SEQ ID NO: 115 - DNA, 663 bp, Artificial Sequence
  Synthetic HMC019 Fc fragment
ccatcatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgatgaaggaggctctgaagaaccactacacacagaagagcctctccctgtctctgggtaaa

SEQ ID NO: 116 - DNA, 663 bp, Artificial Sequence
  Synthetic HMC020 Fc fragment
ccatcatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgctgcatgaggctctgaagaaccactacacacagaagagcctctccctgtctctgggtaaa

SEQ ID NO: 117 - DNA, 663 bp, Artificial Sequence
  Synthetic HMC028 Fc fragment
ccatcatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcacccccctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtggcgtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgatgcatgaggctctgcacaaccactacacacagaagagcctctccctgtctctgggtaaa

SEQ ID NO: 118 - DNA, 663 bp, Artificial Sequence
  Synthetic HMC029 Fc fragment
ccatcatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctctccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtgtcgtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgatgcatgaggctctgcacaaccactacacacagaagagcctctccctgtctctgggtaaa

SEQ ID NO: 119 - DNA, 663 bp, Artificial Sequence
  Synthetic HMC031 Fc fragment
ccatcatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtgtcgtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgatgcatgaggctctgcacagccactacacacagaagagcctctccctgtctctgggtaaa

(19) United States
(12) Patent Application Publication
(10) Pub. No.: US 2014/0357843 A1
(43) Pub. Date: Dec. 4, 2014

(54) IMMUNOGLOBULIN FC VARIANTS

(71) Applicant: HANMI SCIENCE CO., LTD, Hwaseong-si, Gyeonggi-do (KR)

(72) Inventors: Euh Lim Oh, Seoul (KR); Yong Ho Huh, Seoul (KR); Sang Youn Hwang, Hwaseong-si (KR); In Young Choi, Yongin-si (KR); Sung Youb Jung, Suwon-si (KR); Se Chang Kwon, Seoul (KR)

(73) Assignee: HANMI SCIENCE CO., LTD, Hwaseong-si, Gyeonggi-do (KR)

(21) Appl. No.: 14/369,616

(22) PCT Filed: Dec. 28, 2012
(86) PCT No.: PCT/KR2012/011739

§ 371 Date: Jun. 27, 2014

(30) Foreign Application Priority Data
Dec. 30, 2011 (KR) 10-2011-0147683

(51) Int. Cl. A61K 47/48; A61K 38/26; C07K 16/28

(52) U.S. Cl. CPC A61K 47/48561; C07K 16/283; A61K 38/26; C07K 2317/92
USPC 530/387.3; 530/391.7; 530/391.9

(57) ABSTRACT

The present invention relates to immunoglobulin Fc variants having an increased binding affinity for FcRn, which is characterized by including one or more amino acid modifications selected from the group consisting of 307S, 308F, 380S, 380A, 428L, 429K, 430S, 433K and 434S (this numbering is according to the EU index) in the constant region of a native immunoglobulin Fc fragment. Owing to the high binding affinity for FcRn, the immunoglobulin Fc variants according to the present invention show more prolonged in vivo half-life, and thus can be used for the preparation of a long-acting formulation of protein drugs.

### TECHNICAL FIELD

The present invention relates to immunoglobulin Fc variants having an increased binding affinity for FcRn (neonatal Fc receptor) and a method for increasing in vivo half-life of a physiologically active polypeptide using the same. The immunoglobulin Fc variants of the present invention are characterized by including one or more amino acid modifications selected from the group consisting of 307S, 308F, 380S, 380A, 428L, 429K, 430S, 433K and 434S (this numbering is according to the EU index) in the constant region of a native immunoglobulin Fc fragment.

### BACKGROUND ART

An antibody is an immune protein that binds to a particular antigen. An antibody is composed of two light polypeptide chains and two heavy polypeptide chains. Each chain is composed of immunoglobulin domains and has both a variable region and a constant region. The variable regions show significant sequence diversity between the antibodies and are responsible for binding to the target antigen. The constant regions, having relatively low sequence diversity, are responsible for binding to a number of natural proteins and elicit important biochemical events.

Detailed descriptions of the structures, functions and subclasses of antibodies are disclosed in a lot of previous literature (Burton D R: Immunoglobulin G: functional sites, Mol. Immunol. 22: 161-206, 1985). The region between the Fc domains in IgG mediates interaction with the neonatal Fc receptor (FcRn). FcRn catches IgG that enters the cell by endocytosis and recycles it from the endosome back to the bloodstream (Raghavan et al., Annu. Rev. Cell Dev. Biol. 12: 181-220, 1996). With the exclusion of kidney filtration due to the large size of the antibody itself, this process results in prolonged antibody serum half-life ranging from one to three weeks.

Studies of rat and human Fc domains have demonstrated the importance of some Fc residues to the FcRn binding. In the murine Fcγ domain, random mutation and phage display selection at the sites T252, T254, and T256 lead to a triple mutant T252L/T254S/T256F that has increased FcRn binding affinity and serum half-life (Ghetie et al., Nat. Biotech. 15(7): 637-640, 1997). Disruption of the Fc/FcRn interaction by mutations at the sites I253, H310 and H435 also lead to decreased in vivo half-life (Medesan et al., J. Immunol. 158(5): 2211-2217, 1997).

It has been proven that mutations of some residues in the human Fcγ domain that are important for binding to FcRn increase serum half-life. In particular, in human Fcγ1, when three residues are substituted with the other 19 conventional amino acids, some point mutants showed increased FcRn binding affinity (Hinton et al., J. Biol. Chem. 279(8): 6213-6216, 2004). The amino acids of Fc, H310 and H435, and L309 and I253 are known to be mainly involved in the binding to FcRn through a salt bridge and a hydrophobic bond. It was also reported that mutations in T250, M252, S254, T256, V308, E380, M428 and N434 increased or decreased the FcRn-binding affinity (Roopenian et al., Nat. Rview Immunology 7: 715-725, 2007).

Korean Patent No. 10-1027427 discloses Trastuzumab (Herceptin, Genentech) variants having an increased FcRn-binding affinity, and these variants contain one or more amino acid modifications selected from 257C, 257M, 257L, 257N, 257Y, 279Q, 279Y, 308F and 308Y. Korean Patent Publication No. 2010-0099179 provides beacizumab (Avastin, Genentech) variants and these variants show increased in vivo half-life by containing amino acid modifications at N434S, M252Y/M428L, M252Y/N434S and M428L/N434S.

The administration of an antibody protein as a therapeutic requires injections with a prescribed frequency in consideration of the clearance and in vivo half-life properties of the protein. Longer in vivo half-lives allow less frequent injections or a lower dosage, either of which is clearly advantageous. Although the mutations previously reported in the Fc domain yielded some antibody variants with increased FcRn binding affinity and prolonged in vivo half-lives, these mutations were found to not be optimal, and some variants did not enhance in vivo half-life to a satisfactory level.

Meanwhile, the need to increase in vivo half-life is not a problem limited to antibody proteins. Therapeutic proteins, including low molecular weight polypeptides, cytokines and hormones, are easily denatured due to low stability, degraded by proteolytic enzymes in the blood, and finally removed by the action of the kidney or liver. Therefore, protein drugs including polypeptides as pharmaceutically active ingredients should be frequently administered to patients in order to maintain their optimal blood concentration and titer. However, since most protein drugs are administered to patients in injectable formulations, frequent injections for maintaining optimal blood concentration of the active polypeptide cause tremendous pain.

In order to solve these problems, attachment of polymers such as PEG or fusion of proteins such as albumin has been attempted to increase in vivo half-life of a polypeptide drug. However, even though PEG is attached or albumin is fused thereto, the polypeptide drug still shows relatively low biological activity or its in vivo half-life cannot be increased to a sufficient level. Korean Patent No. 10-0567902 discloses a conjugate that is prepared by linking an immunoglobulin fragment with a non-peptidyl polymer in vitro. In this method, only the immunoglobulin fragment is produced in E. coli and then linked to a physiologically active polypeptide through the non-peptidyl polymer, which results in extending the half-life of the polypeptide by minimizing the activity reduction thereof. This method has been recognized as a general technique that can be applied to non-native or synthetic physiologically active substances that are not found in nature as well as to native peptides and proteins. However, there is still a need to maximize the in vivo half-life of therapeutic physiologically active substances including peptides and proteins.

Accordingly, the present inventors have developed immunoglobulin Fc variants having an increased binding affinity for FcRn, compared to native immunoglobulin Fc fragments. They also found that a protein conjugate in which the immunoglobulin Fc variant of the present invention is covalently linked to a physiologically active polypeptide via a non-peptidyl polymer shows more prolonged in vivo half-life due to the increased binding affinity for FcRn.

### DISCLOSURE

### Technical Problem

An object of the present invention is to provide immunoglobulin Fc variants having an increased binding affinity for FcRn.

Another object of the present invention is to provide a protein conjugate comprising the immunoglobulin Fc variant of the present invention.

Still another object of the present invention is to provide a method for increasing in vivo half-life of a physiologically active polypeptide by using the immunoglobulin Fc variant of the present invention.

### Technical Solution

In one aspect, the present invention provides an immunoglobulin Fc variant having an increased binding affinity for FcRn, comprising one or more amino acid modifications selected from the group consisting of 307S, 308F, 380S, 380A, 428L, 429K, 430S, 433K and 434S (this numbering is according to the EU index) in the constant region of a native immunoglobulin Fc fragment.

In another aspect, the present invention provides a protein conjugate having increased in vivo half-life, in which a physiologically active polypeptide is covalently linked to the immunoglobulin Fc variant according to the present invention via a non-peptidyl polymer.

In still another aspect, the present invention provides a method for increasing in vivo half-life of a physiologically active polypeptide, comprising the step of covalently linking the immunoglobulin Fc variant according to the present invention to the physiologically active polypeptide via a non-peptidyl polymer.

### Advantageous Effects

Since the immunoglobulin Fc variants according to the present invention show a high binding affinity for FcRn, they can increase in vivo half-life of a physiologically active polypeptide. Therefore, the protein conjugate having a prolonged in vivo half-life, in which the immunoglobulin Fc variant of the present invention is covalently linked to the physiologically active polypeptide via a non-peptidyl polymer, can be effectively used for the preparation of a long-acting formulation of protein drugs with remarkably low administration frequency.

### DESCRIPTION OF DRAWINGS

FIGS. 1 to 3 are the results of ELISA for analyzing FcRn-binding affinities of the immunoglobulin Fc variants according to the present invention, in which the left graphs represent binding affinity at pH 6.0, and the right graphs represent binding affinity at pH 7.4; and

FIG. 4 are the results of ELISA for analyzing FcRn-binding affinities of the protein conjugates according to the present invention, in which the protein conjugate was prepared by linking the immunoglobulin Fc variant and a physiologically active polypeptide via a non-peptidyl polymer, the left graphs represent binding affinity at pH 6.0, and the right graphs represent binding affinity at pH 7.4.

### BEST MODE

In one aspect of the present invention, the present invention relates to immunoglobulin Fc variants having an increased binding affinity for FcRn, which includes one or more amino acid modifications selected from the group consisting of 307S, 308F, 380S, 380A, 428L, 429K, 430S, 433K and 434S (this numbering is according to the EU index) in the constant region of a native immunoglobulin Fc fragment.

As used herein, the term “FcRn” or “neonatal Fc receptor” means a protein that binds to the IgG antibody Fc region and is encoded at least in part by an FcRn gene. The FcRn may be from any organism including humans, mice, rats, rabbits, and monkeys, but is not limited thereto. As is known in the art, the functional FcRn protein includes two polypeptides, often referred to as the heavy chain and the light chain. The light chain is beta-2-microglobulin and the heavy chain is encoded by the FcRn gene. Unless otherwise indicated herein, FcRn or an FcRn protein refers to the complex of FcRn heavy chain with beta-2-microglobulin.

As used herein, the term “native (wild-type) polypeptide” means a non-modified polypeptide that is subjected to modification to generate a variant. The native polypeptide is a naturally occurring polypeptide or a derivative or a manipulated one thereof. The native polypeptide may refer to the polypeptide as it is, a composition including the same, or an amino acid sequence encoding the same. Therefore, the term “native immunoglobulin”, as used herein, means a non-modified immunoglobulin polypeptide which generates a variant through amino acid modifications. Interchangeably, the term “parent immunoglobulin”, which means a non-modified immunoglobulin polypeptide generating a variant through amino acid modifications, may also be used.

As used herein, the term “amino acid modification” means amino acid substitution, insertion, and/or deletion, preferably substitution in an amino acid sequence. As used herein, the term “amino acid substitution” or “substitution” means the substitution of an amino acid at a particular position in a native polypeptide sequence with another amino acid. For example, an immunoglobulin Fc variant including T307S substitution refers to a variant, in which threonine at position 307 in the amino acid sequence of the native immunoglobulin Fc fragment is substituted with serine.

As used herein, the term “immunoglobulin Fc variant” means to include one or more amino acid modifications, as compared to those of the native immunoglobulin Fc fragment. In a preferred embodiment of the present invention, the immunoglobulin Fc variant means a variant which includes one or more amino acid modifications selected from the group consisting of 307S, 308F, 380S, 380A, 428L, 429K, 430S, 433K and 434S (this numbering is according to the EU index) so as to have an increased binding affinity for FcRn, as compared to the native immunoglobulin Fc fragment.

The present invention is based on the identification of some mutations in the constant region of the immunoglobulin Fc fragment, which show improved binding affinity for FcRn. The present invention provides immunoglobulin Fc variants having an increased binding affinity for FcRn and/or in vivo half-life, as compared to the corresponding native immunoglobulin Fc fragment. The in vivo half-life of an antibody and other physiologically active substances (that is, persistence in the serum or other tissues of a subject) is an important clinical parameter that determines administration dosage and frequency thereof. Therefore, physiologically active substances, including antibodies having a prolonged in vivo half-life, are pharmaceutically important and advantageous.

In this regard, there is a report that the in vivo half-life of an immunoglobulin correlates with the binding of Fc to FcRn. The immunoglobulin Fc fragment is internalized in acidic endosomes after uptake into endothelial cells via non-specific pinocytosis. FcRn binds to the immunoglobulin at the acidic pH (<6.5) of endosomes and releases the same at the basic pH (>7.4) of the bloodstream. Thus, FcRn salvages the immunoglobulin from lysosomal degradation. When a serum immunoglobulin level decreases, more FcRn molecules are available for immunoglobulin binding so that an increased amount of immunoglobulin is salvaged. Conversely, if a serum immunoglobulin level rises, FcRn becomes saturated, thereby increasing the proportion of immunoglobulin that is internalized and degraded (Ghetie and Ward, Annu. Rev. Immunol. 18: 739-766, 2000).

Based on the close relationship between the binding of immunoglobulin Fc fragment to FcRn and in vivo half-life of immunoglobulin, a plurality of mutations are introduced into the constant region of a native immunoglobulin Fc fragment in order to develop immunoglobulin Fc variants that show increased binding affinity for FcRn in a low pH environment but substantially no change in binding affinity in a high pH environment. Consequently, modifying one or more amino acids selected from the group consisting of amino acid residues 307, 308, 380, 428, 429, 430, 433 and 434 (this numbering is according to the EU index) in the constant region of the native immunoglobulin Fc fragment has been found to increase the binding affinity for FcRn.

Accordingly, the present invention provides immunoglobulin Fc variants including one or more amino acid modifications selected from the group consisting of 307S, 308F, 380S, 380A, 428L, 429K, 430S, 433K and 434S (this numbering is according to the EU index) in the constant region of the native immunoglobulin Fc fragment.

In one preferred embodiment, the present invention provides immunoglobulin Fc variants including the amino acid modification selected from the group consisting of 428L/434S, 433K/434S, 429K/433K, 428L/433K, 308F/380A, 307S/380S and 380S/434S in the constant region of the native immunoglobulin Fc fragment.

In a specific embodiment, the present invention provides an immunoglobulin Fc variant in which histidine at position 428 is substituted with lysine and asparagine at position 434 is substituted with serine in the constant region of the native immunoglobulin Fc fragment. The immunoglobulin Fc variant including the amino acid modification of 428L/434S has an amino acid sequence represented by SEQ ID NO: 74, and is encoded by a nucleotide having a base sequence represented by SEQ ID NO: 113. In the present invention, the immunoglobulin Fc variant is designated as HMC002.

In another specific embodiment, the present invention provides an immunoglobulin Fc variant in which histidine at position 433 is substituted with lysine and asparagine at position 434 is substituted with serine in the constant region of the native immunoglobulin Fc fragment. The immunoglobulin Fc variant including the amino acid modification of 433K/434S has an amino acid sequence represented by SEQ ID NO: 80, and is encoded by a nucleotide having a base sequence represented by SEQ ID NO: 114. In the present invention, the immunoglobulin Fc variant is designated as HMC008.

In still another specific embodiment, the present invention provides an immunoglobulin Fc variant in which histidine at position 429 is substituted with lysine and histidine at position 433 is substituted with lysine in the constant region of the native immunoglobulin Fc fragment. The immunoglobulin Fc variant including the amino acid modification of 429K/433K has an amino acid sequence represented by SEQ ID NO: 91, and is encoded by a nucleotide having a base sequence represented by SEQ ID NO: 115. In the present invention, the immunoglobulin Fc variant is designated as HMC019.

In still another specific embodiment, the present invention provides an immunoglobulin Fc variant in which methionine at position 428 is substituted with leucine and histidine at position 433 is substituted with lysine in the constant region of the native immunoglobulin Fc fragment. The immunoglobulin Fc variant, including the amino acid modification of 428L/433K, has an amino acid sequence represented by SEQ ID NO: 92 and is encoded by a nucleotide having a base sequence represented by SEQ ID NO: 116. In the present invention, the immunoglobulin Fc variant is designated as HMC020.

In still another specific embodiment, the present invention provides an immunoglobulin Fc variant in which valine at position 308 is substituted with phenylalanine and glutamic acid at position 380 is substituted with alanine in the constant region of the native immunoglobulin Fc fragment. The immunoglobulin Fc variant, including the amino acid modification of 308F/380A, has an amino acid sequence represented by SEQ ID NO: 100 and is encoded by a nucleotide having a base sequence represented by SEQ ID NO: 117. In the present invention, the immunoglobulin Fc variant is designated as HMC028.

In still another specific embodiment, the present invention provides an immunoglobulin Fc variant in which threonine at position 307 is substituted with serine and glutamic acid at position 380 is substituted with serine in the constant region of the native immunoglobulin Fc fragment. The immunoglobulin Fc variant, including the amino acid modification of 307S/380S, has an amino acid sequence represented by SEQ ID NO: 101, and is encoded by a nucleotide having a base sequence represented by SEQ ID NO: 118. In the present invention, the immunoglobulin Fc variant is designated as HMC029.

In still another specific embodiment, the present invention provides an immunoglobulin Fc variant in which glutamic acid at position 380 is substituted with serine and asparagine at position 434 is substituted with serine in the constant region of the native immunoglobulin Fc fragment. The immunoglobulin Fc variant including the amino acid modification of 380S/434S has an amino acid sequence represented by SEQ ID NO: 103 and is encoded by a nucleotide having a base sequence represented by SEQ ID NO: 119. In the present invention, the immunoglobulin Fc variant is designated as HMC031.

The parent immunoglobulin Fc fragment to be used for the preparation of the above described immunoglobulin Fc variants may be preferably HMC001 produced from an E. coli transformant HM11201 (KCCM-10660P) that is disclosed in Korean Patent No. 10-824505. HMC001 is an immunoglobulin Fc fragment having an amino acid sequence represented by SEQ ID NO: 73.

In the present invention, the immunoglobulin Fc variants are defined according to the amino acid modifications introduced into the parent immunoglobulin Fc fragment and the numbering of the amino acid residues therein is that of the EU index as in Kabat (Kabat et al., Sequence of proteins of immunological interest, 5th Ed., United States Public Health Service, National Institutes of Health, Bethesda, 1991). At this time, since HMC001 used as the parent immunoglobulin Fc fragment in the present invention is an immunoglobulin Fc fragment that is devoid of an initial methionine residue by removal of a part of the N-terminus upon production in the E. coli transformant, it may not be according to the Kabat numbering. HMC001 used as the parent immunoglobulin Fc fragment in the present invention has proline as a first amino acid, and the amino acid numbering of the immunoglobulin Fc variants according to the present invention complies therewith. However, the mutation positions introduced into the immunoglobulin Fc variants of the present invention are not limited to HMC001 and may be defined according to the Kabat numbering. For example, the “81T” of HCM001 is the same as the “307T” according to the Kabat numbering.

The immunoglobulin Fc variants of the present invention show increased binding affinity at low pH, for example, at pH 6.0 of endosomes, but no increased binding affinity at high pH, for example, at pH 7.4. Further, their internalization into endosomes increases, but the immunoglobulin Fc variants of the present invention can be released at a normal rate, resulting in increased in vivo half-life.

In the present invention, the native immunoglobulin Fc fragment may be an Fc fragment derived from human IgG1, IgG2, IgG3 or IgG4. In the present invention, the native immunoglobulin Fc fragment may be preferably an Fc fragment derived from human IgG4, which does not include the variable region and the heavy chain, and is aglycosylated.

The immunoglobulin Fc variants according to the present invention include one or more amino acid modifications, as compared to the native immunoglobulin Fc fragment, and therefore have different amino acid sequences. The amino acid sequences of the immunoglobulin Fc variants according to the present invention are substantially homologous to that of the native immunoglobulin Fc fragment. For example, the amino acid sequences of the immunoglobulin Fc variants according to the present invention may have approximately 80% or higher homology, preferably approximately 90% or higher homology, and most preferably approximately 95% or higher homology than that of the native immunoglobulin Fc fragment. The amino acid modification may be genetically performed by a molecular biological method or may be performed by an enzymatic or chemical method.

The immunoglobulin Fc variants according to the present invention may be prepared by any conventional method known in the art. In one embodiment, the immunoglobulin Fc variants according to the present invention are used to create nucleic acids that encode the polypeptide sequences including particular amino acid modifications, followed by being cloned into host cells, expressed and assayed, if desired. A variety of methods are described in relevant literature (Molecular Cloning—A Laboratory Manual, 3rd Ed., Maniatis, Cold Spring Harbor Laboratory Press, New York, 2001; Current Protocols in Molecular Biology, John Wiley & Sons).

The nucleic acids that encode the immunoglobulin Fc variants according to the present invention may be incorporated into an expression vector for protein expression. Expression vectors typically include a protein operably linked, that is, placed in a functional relationship, with control or regulatory sequences, selectable markers, any fusion partners, and/or additional elements. The immunoglobulin Fc variant according to the present invention may be produced by culturing a host cell transformed with the nucleic acid, preferably an expression vector containing the nucleic acid encoding the immunoglobulin Fc variant, under conditions appropriate so as to induce or cause the expression thereof. A wide variety of appropriate host cells include, but are not limited to, mammalian cells, bacterial cells, insect cells, and yeast cells. The methods of introducing an exogenous nucleic acid into host cells are well known in the art, and will vary with the host cell used. E. coli, which is industrially valuable due to low production costs, can preferably be used as a host cell to produce the immunoglobulin Fc variants according to the present invention.

Therefore, the scope of the present invention includes a method for preparing the immunoglobulin Fc variant, comprising the steps of:

1) culturing the host cells, into which the nucleic acid encoding the immunoglobulin Fc variant is introduced, under the conditions suitable for protein expression; and

2) purifying or isolating the immunoglobulin Fc variant expressed from the host cells.

Antibodies may be isolated or purified by various methods known in the art. Standard purification methods include chromatographic techniques, electrophoresis, immunoprecipitation, dialysis, filtration, concentration, and chromatofocusing techniques. As is well known in the art, a variety of natural proteins such as bacterial proteins A, G, and L can bind to antibodies, and thus these proteins can be used for the purification of antibodies. Purification can often be enabled by using a particular fusion partner. For example, proteins may be purified by using a glutathione resin if a GST fusion is employed, by using a Ni⁺² affinity chromatography if a His-tag is employed, or by using an immobilized anti-flag antibody if a flag-tag is used.

In another aspect, the present invention relates to a protein conjugate having an increased in vivo half-life, in which a physiologically active polypeptide is covalently linked to the immunoglobulin Fc variant of the present invention via a non-peptidyl polymer.

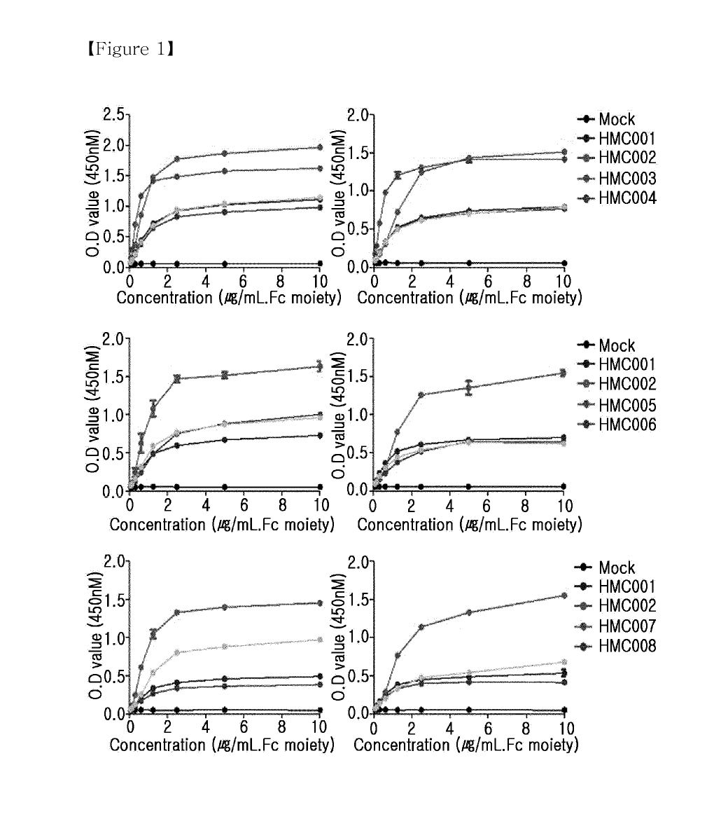

The immunoglobulin Fc variants according to the present invention show increased binding affinity at low pH of endosomes, for example, at pH 6.0, whereas no corresponding increased binding affinity at high pH, for example, at pH 7.4. Thus, their internalization into endosomes increases, but they are released at a normal rate, leading to increased in vivo half-life (see FIG. 1). Therefore, the immunoglobulin Fc variants according to the present invention can be used as a carrier for increasing in vivo half-life of physiologically active polypeptides including protein drugs. Accordingly, the protein conjugate of the present invention can be effectively used in the preparation of a long-acting drug formulation having remarkably increased in vivo half-life due to high binding affinity for FcRn.

Further, the present invention provides a method for preparing a long-acting drug formulation by covalently linking the immunoglobulin Fc variant of the present invention to a physiologically active polypeptide via a non-peptidyl polymer.

The preparation method according to the present invention may comprise the steps of:

1) covalently linking the immunoglobulin Fc variant to the physiologically active polypeptide via the non-peptidyl polymer having a terminal reactive group; and

2) isolating a conjugate in which the physiologically active polypeptide, the non-peptidyl polymer, and the immunoglobulin Fc variant are covalently linked to each other.

The non-peptidyl polymer useful in the present invention may be selected from the group consisting of biodegradable polymers such as polyethylene glycol, polypropylene glycol, a copolymer of ethylene glycol and propylene glycol, polyoxyethylated polyol, polyvinyl alcohol, polysaccharide, dextran, polyvinyl ethyl ether, PLA (polylactic acid) and PLGA (polylactic-glycolic acid), lipopolymers, chitins, hyaluronic acid and combinations thereof, and preferably polyethylene glycol. Also, their derivatives that are known in the art or that can be readily prepared using a conventional technique fall within the scope of the present invention.

The physiologically active polypeptide to be linked to the immunoglobulin Fc variant according to the present invention may be any one without limitation, as long as it is needed to have increased in vivo half-life. For example, various physiologically active polypeptides that are used for the purpose of treating or preventing human diseases, such as cytokines, interleukins, interleukin-binding proteins, enzymes, antibodies, growth factors, transcription factors, blood factors, vaccines, structural proteins, ligand proteins or receptors, cell surface antigens, and receptor antagonists, and derivatives or analogs thereof, may be used.

Examples of the physiologically active polypeptides useful in the present invention include human growth hormones, growth hormone releasing hormones, growth hormone releasing peptides, interferons and interferon receptors (e.g., interferon-alpha, -beta and -gamma, soluble type I interferon receptors), granulocyte colony-stimulating factors (G-CSF), granulocyte-macrophage colony-stimulating factors (GM-CSF), glucagon-like peptides (GLP-1), G-protein-coupled receptors, interleukins (e.g., IL-1 receptors, IL-4 receptors), enzymes (e.g., glucocerebrosidase, iduronate-2-sulfatase, alpha-galactosidase-A, agalsidase alpha, beta, alpha-L-iduronidase, butyrylcholinesterase, chitinase, glutamate decarboxylase, imiglucerase, lipase, uricase, platelet-activatingfactor acetylhydrolase, neutral endopeptidase, myeloperoxidase), interleukin- and cytokine-binding proteins (e.g., IL-18 bp, TNF-binding protein), macrophage activating factors, macrophage peptides, B-cell factors, T-cell factors, Protein A, allergy inhibitors, cell necrosis glycoproteins, immunotoxins, lymphotoxins, tumor necrosis factor, tumor suppressors, transforming growth factor, alpha-1 anti-trypsin, albumin, alpha-lactalbumin, apolipoprotein-E, erythropoietin, highly glycosylated erythropoietin, angiopoietins, hemoglobin, thrombin, thrombin receptors activating peptides, thrombomodulin, blood factor VII, blood factor VIIa, blood factor VIII, blood factor IX and blood factor XIII, plasminogen activators, fibrin-binding peptides, urokinase, streptokinase, hirudin, Protein C, C-reactive protein, renin inhibitor, collagenase inhibitor, superoxide dismutase, leptin, platelet-derived growth factor, epithelial growth factor, epidermal growth factor, angiostatin, endostatin angiotensin, bone growth factor, bone stimulating protein, calcitonin, insulin, atriopeptin, cartilage inducing factor, elcatonin, connective tissue activating factor, tissue factor pathway inhibitor, follicle stimulating hormone, luteinizing hormone, luteinizing hormone releasing hormone, nerve growth factors (e.g., nerve growth factor, cilliary neurotrophic factor, axogenesis factor-1, brain-natriuretic peptide, glial derived neurotrophic factor, netrin, neurophil inhibitory factor, neurotrophic factor, neuturin), parathyroid hormone, relaxin, secretin, somatomedin, insulin-like growth factor, adrenocortical hormone, glucagon, cholecystokinin, pancreatic polypeptide, gastrin releasing peptide, corticotropin releasing factor, thyroid stimulating hormone, autotaxin, lactoferrin, myostatin, receptors (e.g., TNFR(P75), TNFR(P55), IL-1 receptor, VEGF receptor, B-cell activator receptor), receptor antagonists (e.g., IL1-Ra), cell surface antigens (e.g., CD 2, 3, 4, 5, 7, 11a, 11b, 18, 19, 20, 23, 25, 33, 38, 40, 45, 69), monoclonal antibodies, polyclonal antibodies, antibody fragments (e.g., scFv, Fab, Fab′, F(ab′)2 and Fd), and virus derived vaccine antigens, but are not limited thereto. The antibody fragments may be selected from Fab, Fab′, F(ab′)₂, Fd and scFv having an ability to bind to a particular antigen.

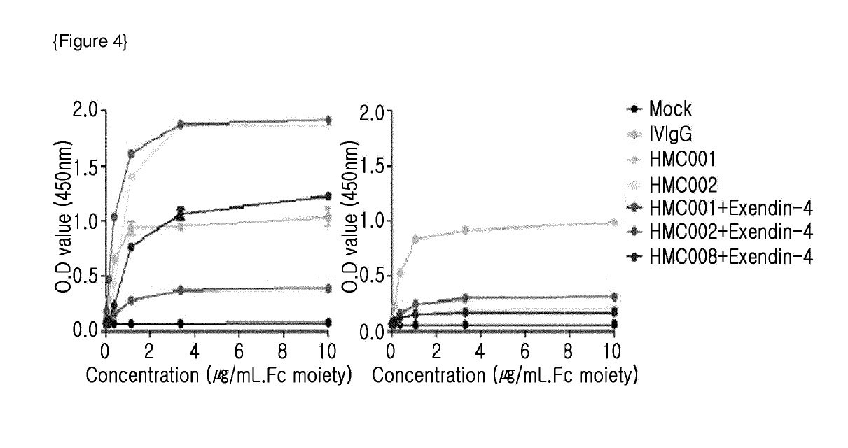

The protein conjugate in which the physiologically active polypeptide is covalently linking to the immunoglobulin Fc variant of the present invention via the non-peptidyl polymer shows remarkably prolonged in vivo half-life due to a high binding affinity for FcRn (see FIG. 4). Therefore, the protein conjugate that is prepared by using the immunoglobulin Fc fragment according to the present invention as a carrier can increase persistence in the blood, and thereby remarkably reduce administration frequency.

### MODE FOR INVENTION

The present invention is further illustrated by the following Examples. However, it shall be understood that these Examples are only used to specifically set forth the present invention, rather than being understood that they are used to limit the present invention in any form.

### Example 1

### Construction of a Vector Expressing an Immunoglobulin Fc Fragment

The positions involved in binding affinity for human FcRn were selected from the amino acid sequence (SEQ ID NO: 73) of HMC001 produced from the E. coli transformant HM11201 (KCCM-10660P, Korean Patent No. 10-0824505), and mutagenesis was induced thereon to substitute the amino acid of the corresponding position with another amino acid so as to increase the binding affinity for FcRn. To achieve this, primers for nucleotide substitution and a QuikChange™ Site-directed Mutagenesis kit (Stratagene) were used. In this regard, the primers used in site-directed mutagenesis are shown in the following Tables 1 to 3.

For site-directed mutagenesis by polymerase chain reaction (PCR), 50 ng of an HMC001 expression vector, each primer pair, dNTP and PfuTurbo™ polymerase (Stratagene) were added in a PCR tube, and reaction was performed as follows: denaturation at 95° C. for 30 seconds, 16 cycles of 95° C. for 30 seconds, 55° C. for 1 minute and 68° C. for 14 minutes, and final extension of 68° C. 5 minutes. PCR products of approximately 1.2 kb amplified above were subjected to base sequence analysis through DNA sequencing, and the results are shown in the following Table 4. The amino acid numbering described in Table 4 is according to the EU index, and the number in parentheses represents the amino acid numbering based on the amino acid sequence of HMC001 (SEQ ID NO: 73).

Each of the amplified PCR products was cleaved with NdeI and BamHI and then inserted into a plasmid pET22b (Novagen) that was previously treated with the same restriction enzymes to thereby obtain expression vectors including each of the immunoglobulin Fc variants.

TABLE 1

| | | | SEQ ID |
|---|---|---|---|
| Variant | Primer | Base sequence | NO |
| HMC002 | FcM202Lss | CTTCTCATGCTCCGTGCTGCATGAG | 1 |
| HMC023 | | GCTCTGCAC | |
| HMC002 | FcM202Las | GTGCAGAGCCTCATGCAGCACGGAG | 2 |
| HMC023 | | CATGAGAAG | |
| HMC002 | FcN208Sss | CATGAGGCTCTGCACAGCCACTACA | 3 |
| HMC006 | | CACAGAAG | |
| HMC026 | | | |
| HMC031 | | | |
| HMC036 | | | |
| HMC037 | | | |
| HMC038 | | | |
| HMC040 | | | |
| HMC002 | FcN208Sas | CTTCTGTGTGTAGTGGCTGTGCAGA | 4 |
| HMC006 | | GCCTCATG | |
| HMC026 | | | |
| HMC031 | | | |
| HMC036 | | | |
| HMC037 | | | |
| HMC038 | | | |
| HMC040 | | | |
| HMC003 | FcI27Fss | CAAGGACACCCTCATGTTCTCCCGG | 5 |
| | | ACCCCTGAG | |
| HMC003 | FcI27Fas | CTCAGGGGTCCGGGAGAACATGAGG | 6 |
| | | GTGTCCTTG | |
| HMC004 | FcH207Tss | GATGCATGAGGCTCTGACCAACCAC | 7 |
| | | TACACACAG | |
| HMC004 | FcH207Tas | CTGTGTGTAGTGGTTGGTCAGAGCC | 8 |
| | | TCATGCATC | |
| HMC005 | FcH207Kss | GATGCATGAGGCTCTGAAGAACCAC | 9 |
| | | TACACACAG | |
| HMC005 | FcH207Kas | CTGTGTGTAGTGGTTCTTCAGAGCC | 10 |
| | | TCATGCATC | |
| HMC006 | FcL83Fss | CAGCGTCCTCACCGTCTTCCACCAG | 11 |
| HMC011 | | GACTGGCTG | |
| HMC006 | FcL83Fas | CAGCCAGTCCTGGTGGAAGACGGTG | 12 |
| HMC011 | | AGGACGCTG | |
| HMC007 | FcI27Lss | CAAGGACACCCTCATGCTCTCCCGG | 13 |
| | | ACCCCTGAG | |
| HMC007 | FcI27Las | CTCAGGGGTCCGGGAGAGCATGAGG | 14 |
| | | GTGTCCTTG | |
| HMC008 | FcH207K/N208Sss | GATGCATGAGGCTCTGAAGAGCCAC | 15 |
| | | TACACACAG | |
| HMC008 | FcH207K/N208Sas | CTGTGTGTAGTGGCTCTTCAGAGCC | 16 |
| | | TCATGCATC | |
| HMC009 | FcN208Tss | CATGAGGCTCTGCACACCCACTACA | 17 |
| | | CACAGAAG | |
| HMC009 | FcN208Tas | CTTCTGTGTGTAGTGGGTGTGCAGA | 18 |
| | | GCCTCATG | |
| HMC010 | FcH207T/N208Sss | GATGCATGAGGCTCTGACCAGCCAC | 19 |
| | | TACACACAG | |
| HMC010 | FcH207T/N208Sas | CTGTGTGTAGTGGCTGGTCAGAGCC | 20 |
| | | TCATGCATC | |
| HMC012 | FcE204Rss | ATGCTCCGTGATGCATCGGGCTCTG | 21 |
| | | CACAACCAC | |
| HMC012 | FcE204Ras | GTGGTTGTGCAGAGCCCGATGCATC | 22 |
| | | ACGGAGCAT | |
| HMC013 | FcE204Kss | ATGCTCCGTGATGCATAAGGCTCTG | 23 |
| | | CACAACCAC | |
| HMC013 | FcE204Kas | GTGGTTGTGCAGAGCCTTATGCATC | 24 |
| | | ACGGAGCAT | |

TABLE 2

| | | | SEQ ID |
|---|---|---|---|
| Variant | Primer | Base sequence | NO |
| HMC014 | FcE204R/H207Kss | TGCTCCGTGATGCATCGGGCTCTGA | 25 |
| | | AGAACCACTACACACAG | |
| HMC014 | FcE204R/H207Kas | CTGTGTGTAGTGGTTCTTCAGAGCC | 26 |
| | | CGATGCATCACGGAGCA | |
| HMC015 | FcE204K/H207Kss | TGCTCCGTGATGCATAAGGCTCTGA | 27 |
| | | AGAACCACTACACACAG | |
| HMC015 | FcE204K/H207Kas | CTGTGTGTAGTGGTTCTTCAGAGCC | 28 |
| | | TTATGCATCACGGAGCA | |
| HMC016 | FcH203Rss | CTCATGCTCCGTGATGCGTGAGGCT | 29 |
| | | CTGCACAAC | |
| HMC016 | FcH203Ras | GTTGTGCAGAGCCTCACGCATCACG | 30 |
| | | GAGCATGAG | |
| HMC017 | FcH203Kss | CTCATGCTCCGTGATGAAGGAGGCT | 31 |
| | | CTGCACAAC | |
| HMC017 | FcH203Kas | GTTGTGCAGAGCCTCCTTCATCACG | 32 |
| | | GAGCATGAG | |
| HMC018 | FcH203R/H207Kss | CATGCTCCGTGATGCGTGAGGCTCT | 33 |
| | | GAAGAACCACTACACACAG | |
| HMC018 | FcH203R/H207Kas | CTGTGTGTAGTGGTTCTTCAGAGCC | 34 |
| | | TCACGCATCACGGAGCATG | |
| HMC019 | FcH203K/H207Kss | CATGCTCCGTGATGAAGGAGGCTCT | 35 |
| | | GAAGAACCACTACACACAG | |
| HMC019 | FcH203K/H207Kas | CTGTGTGTAGTGGTTCTTCAGAGCC | 36 |
| | | TCCTTCATCACGGAGCATG | |
| HMC020 | FcM202L/H207Kss | CTTCTCATGCTCCGTGCTGCATGAG | 37 |
| | | GCTCTGAAGAACCACTACACACAC | |
| HMC020 | FcM202L/H207Kas | CTGTGTGTAGTGGTTCTTCAGAGCC | 38 |
| | | TCATGCAGCACGGAGCATGAGAAG | |
| HMC021 | FcH203K/N208Tss | CTCATGCTCCGTGATGAAGGAGGCT | 39 |
| | | CTGCACACCCACTACACACAGAAG | |
| HMC021 | FcH203K/N208Tas | CTTCTGTGTGTAGTGGGTGTGCAG | 40 |
| | | AGCCTCCTTCATCACGGAGCATGAG | |
| HMC022 | FcT81Ess | GTGGTCAGCGTCCTCGAAGTCCTGC | 41 |
| HMC023 | | ACCAGGAC | |
| HMC022 | FcT81Eas | GTCCTGGTGCAGGACTTCGAGGACG | 42 |
| HMC023 | | CTGACCAC | |
| HMC024 | FcE154Ass | AGCGACATCGCCGTGGCGTGGGAGA | 43 |
| HMC026 | | GCAATGGG | |
| HMC028 | | | |
| HMC024 | FcE154Aas | CCCATTGCTCTCCCACGCCACGGCG | 44 |
| HMC026 | | ATGTCGCT | |
| HMC028 | | | |

TABLE 3

| | | | SEQ ID |
|---|---|---|---|
| Variant | Primer | Base sequence | NO |
| HMC025 | FcE154Sss | AGCGACATCGCCGTGTCGTGGGAGA | 45 |
| HMC029 | | GCAATGGG | |
| HMC031 | | | |
| HMC025 | FcE154Sas | CCCATTGCTCTCCCACGACACGGCG | 46 |
| HMC029 | | ATGTCGCT | |
| HMC031 | | | |
| HMC027 | FcV82Pss | GTCAGCGTCCTCACCCCCCTGCACC | 47 |
| HMC028 | | AGGACTGG | |
| HMC027 | FcV82Pas | CCAGTCCTGGTGCAGGGGGGTGAGG | 48 |
| HMC028 | | ACGCTGAC | |
| HMC027 | FcE204Sss | TGCTCCGTGATGCATTCGGCTCTGC | 49 |
| | | ACAACCAC | |
| HMC027 | FcE204Sas | GTGGTTGTGCAGAGCCGAATGCATC | 50 |
| | | ACGGAGCA | |
| HMC029 | FcT81Sss | GTGGTCAGCGTCCTCTCCGTCCTGC | 51 |
| | | ACCAGGAC | |
| HMC029 | FcT81Sas | GTCCTGGTGCAGGACGGAGAGGACG | 52 |
| | | CTGACCAC | |
| HMC030 | FcH203A/E204Sss | TCATGCTCCGTGATGGCTTCGGCTC | 53 |
| | | TGCACAACC | |
| HMC030 | FcH203A/E204Sas | GGTTGTGCAGAGCCGAAGCCATCAC | 54 |
| | | GGAGCATGA | |
| HMC032 | FcH207A/N208Sss | GATGCATGAGGCTCTGGCCAGCCAC | 55 |
| | | TACACACAG | |
| HMC032 | FcH207A/N208Sas | CTGTGTGTAGTGGCTGGCCAGAGCC | 56 |
| | | TCATGCATC | |
| HMC033 | FcE204A/N208Sss | GCTCCGTGATGCATGCGGCTCTGCA | 57 |
| | | CAGCCAC | |
| HMC033 | FcE204A/N208Sas | GTGGCTGTGCAGAGCCGCATGCATC | 58 |
| | | ACGGAGC | |
| HMC034 | FcH203A/N208Sss | CTCATGCTCCGTGATGGCTGAGGCT | 59 |
| | | CTGCACAGC | |
| HMC034 | FcH203A/N208Sas | GCTGTGCAGAGCCTCAGCCATCACG | 60 |
| | | GAGCATGAG | |
| HMC035 | FcM202A/N208Sss | CTTCTCATGCTCCGTGGCGCATGAG | 61 |
| | | GCTCTGCAC | |
| HMC035 | FcM202A/N208Sas | GTGCAGAGCCTCATGCGCCACGGAG | 62 |
| | | CATGAGAAG | |
| HMC036 | FcY93Lss | GCTGAATGGCAAGGAGCTCAAGTGC | 63 |
| | | AAGGTCTCC | |
| HMC036 | FcY93Las | GGAGACCTTGCACTTGAGCTCCTTG | 64 |
| | | CCATTCAGC | |
| HMC037 | FcV82Lss | GGTCAGCGTCCTCACCCTCCTGCAC | 65 |
| | | CAGGACTG | |
| HMC037 | FcV82Las | CAGTCCTGGTGCAGGAGGGTGAGGA | 66 |
| | | CGCTGACC | |
| HMC038 | FcT81Ass | GTGTGGTCAGCGTCCTCGCCGTCCT | 67 |
| | | GCACCAGGA | |
| HMC038 | FcT81Aas | TCCTGGTGCAGGACGGCGAGGACGC | 68 |
| | | TGACCACAC | |
| HMC039 | FcH207L/N208Sss | GATGCATGAGGCTCTGCTCAGCCAC | 69 |
| | | TACACACAG | |
| HMC039 | FcH207L/N208Sas | CTGTGTGTAGTGGCTGAGCAGAGCC | 70 |
| | | TCATGCATC | |
| HMC040 | FcY93A/N208Sss | GCTGAATGGCAAGGAGGCCAAGTGC | 71 |
| | | AAGGTCTCC | |
| HMC040 | FcY93A/N208Sa | GGAGACCTTGCACTTGGCCTCCTTG | 72 |
| | | CCATTCAGC | |

TABLE 4

| Modifi- | | | | | | | | | | | | |
|---|---|---|---|---|---|---|---|---|---|---|---|---|
| cation | | | | | | | | | | | | SEQ |
| positi | 253 | 307 | 308 | 309 | 319 | 380 | 428 | 429 | 430 | 433 | 434 | ID |
| on* | (27) | (81) | (82) | (83) | (93) | (154) | (202) | (203) | (204) | (207) | (208) | NO |
| Modified | Ile | Thr | Val | Leu | Tyr | Glu | Met | His | Glu | His | Asn | |
| AA | | | | | | | | | | | | |
| HMC | | | | | | | Leu | | | | Ser | 74 |
| 002 | | | | | | | | | | | | |
| HMC | Phe | | | | | | | | | | | 75 |
| 003 | | | | | | | | | | | | |
| HMC | | | | | | | | | | Thr | | 76 |
| 004 | | | | | | | | | | | | |
| HMC | | | | | | | | | | Lys | | 77 |
| 005 | | | | | | | | | | | | |
| HMC | | | | Phe | | | | | | | Ser | 78 |
| 006 | | | | | | | | | | | | |
| HMC | Leu | | | | | | | | | | | 79 |
| 007 | | | | | | | | | | | | |
| HMC | | | | | | | | | | Lys | Ser | 80 |
| 008 | | | | | | | | | | | | |
| HMC | | | | | | | | | | | Thr | 81 |
| 009 | | | | | | | | | | | | |
| HMC | | | | | | | | | | Thr | Ser | 82 |
| 010 | | | | | | | | | | | | |
| HMC | | | | Phe | | | | | | | | 83 |
| 011 | | | | | | | | | | | | |
| HMC | | | | | | | | | Arg | | | 84 |
| 012 | | | | | | | | | | | | |
| HMC | | | | | | | | | Lys | | | 85 |
| 013 | | | | | | | | | | | | |
| HMC | | | | | | | | | Arg | Lys | | 86 |
| 014 | | | | | | | | | | | | |
| HMC | | | | | | | | | Lys | Lys | | 87 |
| 015 | | | | | | | | | | | | |
| HMC | | | | | | | | Arg | | | | 88 |
| 016 | | | | | | | | | | | | |
| HMC | | | | | | | | Lys | | | | 89 |
| 017 | | | | | | | | | | | | |
| HMC | | | | | | | | Arg | | Lys | | 90 |
| 018 | | | | | | | | | | | | |
| HMC | | | | | | | | Lys | | Lys | | 91 |
| 019 | | | | | | | | | | | | |
| HMC | | | | | | | Leu | | | Lys | | 92 |
| 020 | | | | | | | | | | | | |
| HMC | | | | | | | | Lys | | | Thr | 93 |
| 021 | | | | | | | | | | | | |
| HMC | | Glu | | | | | | | | | | 94 |
| 022 | | | | | | | | | | | | |
| HMC | | Glu | | | | | Leu | | | | | 95 |
| 023 | | | | | | | | | | | | |
| HMC | | | | | | Ala | | | | | | 96 |
| 024 | | | | | | | | | | | | |
| HMC | | | | | | Ser | | | | | | 97 |
| 025 | | | | | | | | | | | | |
| HMC | | | | | | Ala | | | | | Ser | 98 |
| 026 | | | | | | | | | | | | |
| HMC | | | Phe | | | | | | Ser | | | 99 |
| 027 | | | | | | | | | | | | |
| HMC | | | Phe | | | Ala | | | | | | 100 |
| 028 | | | | | | | | | | | | |
| HMC | | Ser | | | | Ser | | | | | | 101 |
| 029 | | | | | | | | | | | | |
| HMC | | | | | | | | Ala | Ser | | | 102 |
| 030 | | | | | | | | | | | | |
| HMC | | | | | | Ser | | | | | Ser | 103 |
| 031 | | | | | | | | | | | | |
| HMC | | | | | | | | | | Ala | Ser | 104 |
| 032 | | | | | | | | | | | | |
| HMC | | | | | | | | | Ala | | Ser | 105 |
| 033 | | | | | | | | | | | | |
| HMC | | | | | | | | Ala | | | Ser | 106 |
| 034 | | | | | | | | | | | | |
| HMC | | | | | | | Ala | | | | Ser | 107 |
| 035 | | | | | | | | | | | | |
| HMC | | | | | Leu | | | | | | Ser | 108 |
| 036 | | | | | | | | | | | | |
| HMC | | | Pro | | | | | | | | Ser | 109 |
| 037 | | | | | | | | | | | | |
| HMC | | Ala | | | | | | | | | Ser | 110 |
| 038 | | | | | | | | | | | | |
| HMC | | | | | | | | | | Leu | Ser | 111 |
| 039 | | | | | | | | | | | | |
| HMC | | | | | | | | | | Ser | Ser | 112 |
| 040 | | | | | | | | | | | | |

### Example 2

### Production of Immunoglobulin Fc Variants in E. coli

The expression vectors of immunoglobulin Fc variants prepared in Example 1 were transformed into E. coli BL21 (DE3) competent cells (Invitrogen) by heat shock at 42° C. for 1 minute, respectively, followed by culturing on LB solid media supplemented with ampicillin to select colonies resistant to ampicillin.

Thus selected colonies were inoculated in 500 ml of 2×LB media (containing ampicillin), and cultured in a shaking incubator at 37° C. and 120 rpm for 15 hours. After 100 ml of the culture solution was transferred to 500 ml of fresh 2×LB media (containing ampicillin), the resulting solution was incubated until the optical density at 600 nm (OD600) reached 4 to 5. 200 ml of the culture solution was transferred to a 5 L-fermentor at a ratio of 10% (v/v), 2×LB media (containing ampicillin) was injected thereto, and a semi-pH stat fed-batch culture was performed under the conditions of 37° C., 500-700 rpm and an aeration rate of 1 vvm for 45 hours. During fermentation, when the OD600 reached 90 or higher, IPTG (isopropyl-1-thio-β-D-galactopyranoside) was added to the fermentor as an inducer at a final concentration of 0.1 mM so as to induce protein synthesis.

### Example 3

### Isolation and Purification of Immunoglobulin Fc Variants

The culture solution fermented in Example 2 was centrifuged at 12,000 g for 30 minutes to recover a cell pellet. Thus recovered cell pellet was suspended in 10× volumes of a lysis buffer (20 mM Tris (pH 9.0), 1 mM EDTA (pH 8.0), 0.2 M NaCl, 0.5% triton X-100), and then disrupted three times using a microfluidizer (Microfluidics) at a pressure of 15,000 psi. Thus obtained cell lysate solution was centrifuged at 6,000 g for 30 minutes to obtain only inclusion bodies of the proteins expressed by the E. coli transformant.

The inclusion bodies were washed with 0.5% triton X-100 and distilled water and suspended in an 8 M urea solution containing 10× volumes of 20 mM Tris (pH 9.0) for 2 hours to dissolve them. In order to separate insoluble solid impurities, the inclusion body-dissolved solution was centrifuged at 12,000 g for 30 minutes to collect a supernatant. Then, L-cysteine was added to the supernatant at a final concentration of 1 mM and incubated at room temperature for 1 hour to induce protein reduction.

For refolding of the reduced protein, the inclusion bodies dissolved at 4° C. for 24 hours were diluted with 100× volumes of a refolding solution (2 M urea, 0.25 M arginine, 50 mM Tris (pH 8.5), 0.5 mM Cys)

Thus refolded proteins were purified in an Akta purifier (Amersham Pharmacia Biotech) using an ion exchange column (IEX column, GE Healthcare) to thereby obtain immunoglobulin Fc variants with a purity of 98% or higher.

### Example 4

### Evaluation of Human FcRn-Binding Affinity of Immunoglobulin Fc Variants

To evaluate FcRn-binding affinity of the immunoglobulin Fc variant isolated and purified in Example 3, ELISA was performed. In the following experiment, human FcRn (hFcRn, human neonatal Fcγ receptor) was to be expressed in 293T cells and purified therefrom, and a GST antibody was purchased from Merk Milipore. The FcRn-binding affinity measured in each of the immunoglobulin Fc variants was compared with those of HMC001 and native IgG as a control group.

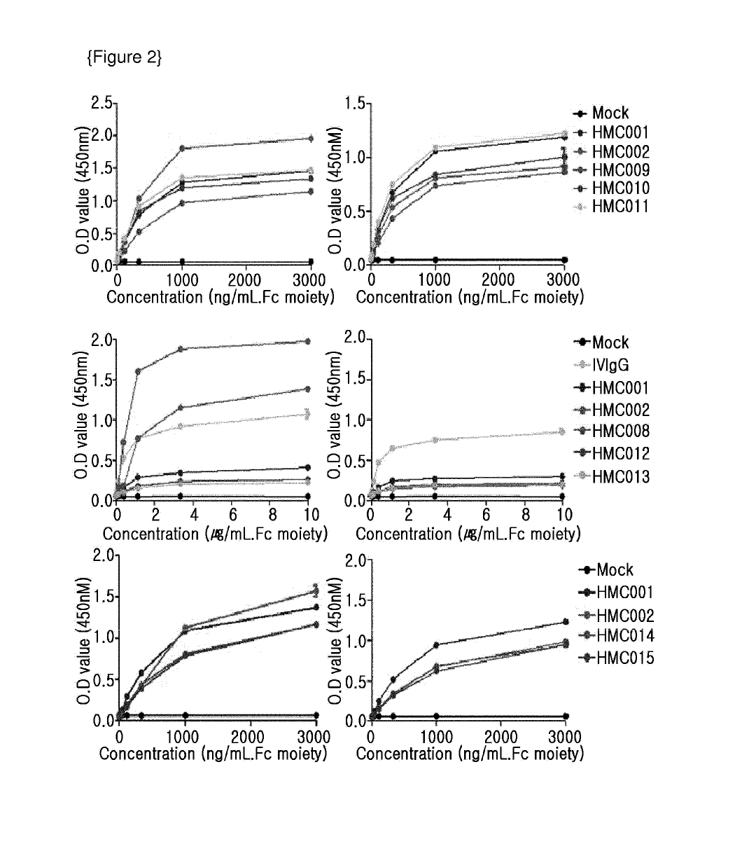

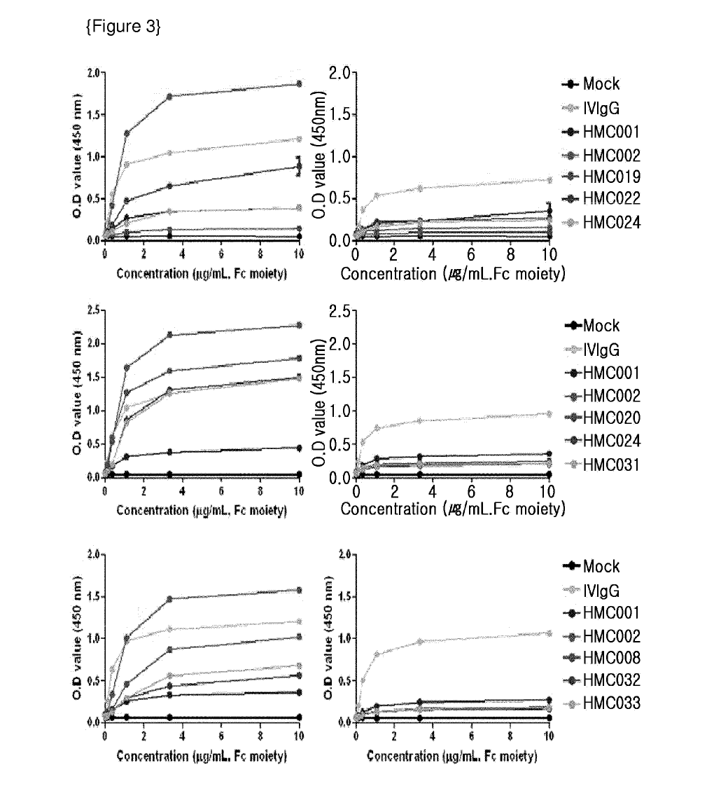

After a 96-well plate was coated with 30 μg/ml of native IgG and each 10 μg/ml of HMC001 and the immunoglobulin Fc variant, 100 μl of 5 μg/ml FcRn was added to each well. For evaluation of binding and dissociation, the experiment was performed at pH 6.0 and 7.4. An assay buffer and a washing buffer used in this experiment have the following composition as shown in Table 5, and the results are shown in FIGS. 1 to 3.

TABLE 5

| | Composition |
|---|---|
| Assay buffer | Sodium phosphate (pH 6.0/7.4), 0.5% BSA, 0.05% |
| | Tween 20 |
| Washing buffer | Sodium phosphate (pH 6.0/7.4), 0.05% Tween 20 |

As shown in FIGS. 1 to 3, the immunoglobulin Fc variants prepared according to the present invention were found to show low FcRn-binding affinity at pH 7.4, but high FcRn-binding affinity at pH 6.0, as compared to HMC001 and native IgG.

### Example 5

### Evaluation of Human FcRn-Binding Affinity of a Conjugate Comprising the Immunoglobulin Fc Variant and Exendin-4

In order to examine whether the immunoglobulin Fc variants according to the present invention show increased binding affinity for human FcRn even in a conjugated form with a protein drug, the immunoglobulin Fc variants, HMC002 and HMC008 that were found to show high FcRn-binding affinity in Example 4 were covalently linked to exendin-4 using PEG having aldehyde reactive groups at both ends, to thereby prepare protein conjugates. Preparation of the protein conjugates was performed in accordance with the method described in Korean Patent No. 10-1058290. ELISA was performed according to the same method as described in Example 4 to evaluate human FcRn-binding affinity of the protein conjugates prepared above.

As shown in FIG. 4, it was found that the immunoglobulin Fc variants according to the present invention maintained high FcRn-binding affinity, even though each of them was linked to a physiologically active polypeptide via a non-peptidyl polymer.

Therefore, the drug conjugate prepared by using the immunoglobulin Fc variant according to the present invention as a carrier has greatly prolonged in vivo half-life owing to the increased binding affinity for FcRn, and thus it can be used as a long-acting drug formulation capable of remarkably reducing administration frequency.

Specific terms used in the present description are given only to describe specific embodiments and are not intended to limit the present invention. Singular forms used in the present description include plural forms unless they apparently represent opposite meanings. The meaning of “including” or “having” used in the present description is intended to embody specific properties, regions, integers, steps, operations, elements and/or components, but is not intended to exclude presence or addition of other properties, regions, integers, steps, operations, elements, components and/or groups.

### INDUSTRIAL APPLICABILITY

The immunoglobulin Fc variants of the present invention show a high binding affinity for FcRn, and thus can increase in vivo half-life of a physiologically active polypeptide. Therefore, the protein conjugate having a prolonged in vivo half-life, in which the immunoglobulin Fc variant of the present invention is covalently linked to the physiologically active polypeptide via a non-peptidyl polymer, can be effectively used for the preparation of a long-acting formulation of protein drugs with remarkably low administration frequency.

1.-29. (canceled)

30. An immunoglobulin Fc variant having an increased binding affinity for FcRn, comprising one or more amino acid modifications selected from the group consisting of 307S, 308F, 380S, 380A, 428L, 429K, 430S, 433K and 434S (this numbering is according to the EU index) in the constant region of a native immunoglobulin Fc fragment.

31. The immunoglobulin Fc variant according to claim 30, wherein the amino acid modification is selected from the group consisting of 428L/434S, 433K/434S, 429K/433K, 428L/433K, 308F/380A, 307S/380S and 380S/434S.

32. The immunoglobulin Fc variant according to claim 30, wherein the immunoglobulin Fc variant comprises the amino acid modification that histidine at position 428 is substituted with lysine and asparagine at position 434 is substituted with serine in the constant region of the native immunoglobulin Fc fragment, and has an amino acid sequence represented by SEQ ID NO: 74.

33. The immunoglobulin Fc variant according to claim 30, wherein the immunoglobulin Fc variant comprises the amino acid modification that histidine at position 433 is substituted with lysine and asparagine at position 434 is substituted with serine in the constant region of the native immunoglobulin Fc fragment, and has an amino acid sequence represented by SEQ ID NO: 80.

34. The immunoglobulin Fc variant according to claim 30, wherein the immunoglobulin Fc variant comprises the amino acid modification that histidine at position 429 is substituted with lysine and histidine at position 433 is substituted with lysine in the constant region of the native immunoglobulin Fc fragment, and has an amino acid sequence represented by SEQ ID NO: 91.

35. The immunoglobulin Fc variant according to claim 30, wherein the immunoglobulin Fc variant comprises the amino acid modification that methionine at position 428 is substituted with leucine and histidine at position 433 is substituted with lysine in the constant region of the native immunoglobulin Fc fragment, and has an amino acid sequence represented by SEQ ID NO: 92.

36. The immunoglobulin Fc variant according to claim 30, wherein the immunoglobulin Fc variant comprises the amino acid modification that valine at position 308 is substituted with phenylalanine and glutamic acid at position 380 is substituted with alanine in the constant region of the native immunoglobulin Fc fragment, and has an amino acid sequence represented by SEQ ID NO: 100.

37. The immunoglobulin Fc variant according to claim 30, wherein the immunoglobulin Fc variant comprises the amino acid modification that threonine at position 307 is substituted with serine and glutamic acid at position 380 is substituted with serine in the constant region of the native immunoglobulin Fc fragment, and has an amino acid sequence represented by SEQ ID NO: 101.

38. The immunoglobulin Fc variant according to claim 30, wherein the immunoglobulin Fc variant comprises the amino acid modification that glutamic acid at position 380 is substituted with serine and asparagine at position 434 is substituted with serine in the constant region of the native immunoglobulin Fc fragment, and has an amino acid sequence represented by SEQ ID NO: 103.

39. The immunoglobulin Fc variant according to claim 30, wherein the native immunoglobulin Fc fragment is selected from the group consisting of Fc fragments of IgG1, IgG2, IgG3 and IgG4.

40. The immunoglobulin Fc variant according to claim 39, wherein the native immunoglobulin Fc fragment is an IgG4 Fc fragment.

41. The immunoglobulin Fc variant according to claim 40, wherein the native immunoglobulin Fc fragment is a human aglycosylated IgG4 Fc fragment.

42. The immunoglobulin Fc variant according to claim 30, wherein the native immunoglobulin Fc fragment has an amino acid sequence represented by SEQ ID NO: 75.

43. The immunoglobulin Fc variant according to claim 30, wherein the immunoglobulin Fc variant does not include the variable region and the light chain of the immunoglobulin.

44. The immunoglobulin Fc variant according to claim 30, wherein the immunoglobulin Fc variant is produced in animal cells or E. coli.

45. The immunoglobulin Fc variant according to claim 44, wherein the immunoglobulin Fc variant is produced in E. coli.

46. The immunoglobulin Fc variant according to claim 30, wherein the immunoglobulin Fc variant shows low FcRn-binding affinity at pH 7.4, but high FcRn-binding affinity at pH 6.0, as compared to the native immunoglobulin Fc fragment.

47. A protein conjugate having increased in vivo half-life, in which a physiologically active polypeptide is covalently linked to the immunoglobulin Fc variant according to claim 30 via a non-peptidyl polymer.

48. The protein conjugate according to claim 47, wherein the non-peptidyl polymer is selected from the group consisting of a poly(ethylene glycol) monopolymer, a poly(propylene glycol) monopolymer, an ethylene glycol-propylene glycol copolymer, polyoxyethylated polyol, polyvinyl alcohol, polysaccharide, dextran, polyvinyl ethyl ether, biodegradable polymers, lipopolymers, chitins, hyaluronic acid and combinations thereof.

49. The protein conjugate according to claim 47, wherein the physiologically active polypeptide is selected from the group consisting of human growth hormones, growth hormone releasing hormones, growth hormone releasing peptides, interferons, colony stimulating factors, interleukins, soluble interleukin receptors, soluble TNF receptors, glucocerebrosidase, macrophage activating factor, macrophage peptide, B cell factor, T cell factor, protein A, allergy inhibitor, cell necrosis glycoproteins, immunotoxin, lymphotoxin, tumor necrosis factor, tumor suppressors, metastasis growth factor, alpha-1 antitrypsin, albumin, apolipoprotein-E, erythropoietin, highly glycosylated erythropoietin, blood factor VII, blood factor VIII, blood factor IX, plasminogen activating factor, urokinase, streptokinase, protein C, C-reactive protein, renin inhibitor, collagenase inhibitor, superoxide dismutase, leptin, platelet-derived growth factor, epidermal growth factor, bone growth factor, bone stimulating protein, calcitonin, insulin, insulin variants, glucagon, glucagon like peptide-1, atriopeptin, cartilage inducing factor, connective tissue activating factor, follicle stimulating hormone, luteinizing hormone, luteinizing hormone releasing hormone, nerve growth factors, parathyroid hormone, relaxin, secretin, somatomedin, insulin-like growth factor, adrenocortical hormone, cholecystokinin, pancreatic polypeptide, gastrin releasing peptide, corticotropin releasing factor, thyroid stimulating hormone, receptors, receptor antagonists, cell surface antigens, monoclonal antibodies, polyclonal antibodies, antibody fragments, and virus derived vaccine antigens.

50. A method for increasing in vivo half-life of a physiologically active polypeptide, comprising the step of covalently linking the immunoglobulin Fc variant according to claim 30 to the physiologically active polypeptide via a non-peptidyl polymer.
